(19) United States
(12) Patent Application Publication
(10) Pub. No.: US 2005/0159709 A1
(43) Pub. Date: Jul. 21, 2005

(54) Safety shield system for a plastic syringe

(75) Inventor: Bradley Wilkinson, North Haledon, NJ (US)

Correspondence Address: COHEN, PONTANI, LIEBERMAN & PAVANE, 551 FIFTH AVENUE, SUITE 1210, NEW YORK, NY, 10176, US

(73) Assignee: Becton, Dickinson and Company

(21) Appl. No.: 10/760,881

(22) Filed: Jan. 20, 2004

(51) Int. Cl. A61M 5/32

(52) U.S. Cl. 604/197

(57) ABSTRACT

A medical device for delivering a medicament to a patient includes a syringe assembly having a barrel defining a reservoir for the medicament, a needle coupled to the forward end of the barrel, and a plunger having a stopper positioned in the reservoir. The plunger is movable within the reservoir to urge the medicament to be expelled from the reservoir. An outer shield having a forward end and a rear end is movable on the barrel from a first position to a second position in which the forward tip of the needle is positioned between the forward and rear ends of the outer shield. An urging member is connected to the outer shield. The plunger interacts with the outer shield to charge the urging member when the needle cannula is inserted into the patient for moving the front of the outer shield toward the second position when the needle is removed from the patient.

### BACKGROUND OF THE INVENTION

1. Field of the Invention

The present invention relates to a prefilled medical device for delivering a dose of medicament by injection and having an integral shield system for preventing accidental needle sticks after use. More particularly, the present invention is directed to a syringe assembly including a safety shield system.

2. Description of the Related Art

Syringes used for the delivery of medicaments to patients are well known. Oftentimes syringes are prefilled with a dosage of a medicament or other substance by a pharmaceutical manufacturer and then distributed to end users such as health care professionals or patients for administration of the prefilled medicament. Such syringes typically include a cylindrical hollow barrel which may be formed of a glass or plastic material and which includes the medicament. One end of the barrel is fitted with a fixed or removable hollow needle, and the other end of the barrel receives a plunger having a stopper which is slidable with respect to the barrel for delivery of the medicament to the hollow needle, i.e., to urge the medicament toward and out of the needle. A syringe assembly, which typically includes the above-described components, is usually stored with a removable needle cover which protects the needle from damage during storage and handling. Prior to use, the needle cover is removed to expose the needle.

To prevent a syringe user and, in particular, a health care professional from inadvertent sticks by the needle after use of the syringe on a patient, the syringe assembly may incorporate a safety shield which forms a guard to cover the needle after use. Certain attributes to be considered in such syringe assemblies are that the shield should be intuitive and easy to use, should preferably provide consistent and reliable shield deployment, and should be operable with one hand. Other attributes are that such syringe assemblies require no change in current medicament delivery techniques, allow for dose adjustment, are preferably autoclavable, and allow for the inspection of contents before and after activation of the shield. Moreover, the use of the shield must not detrimentally affect processing and filling of the syringe at the pharmaceutical company, the assembly (i.e., syringe assembly and safety shield) must be easy to manufacture, must prevent accidental activation, and must limit the possibility of incurring cosmetic or structural damages.

### SUMMARY OF THE INVENTION

The present invention relates to a syringe assembly incorporating a safety shield for covering the needle of the syringe assembly after administration of a dosage of medicament. The safety shield is automatically activated upon full delivery of the medicament dosage in the syringe.

According to the present invention, a medical device for delivering a medicament to a patient includes a syringe assembly having a barrel with a forward end and a rear end and defining a reservoir within which the medicament may be contained. A needle cannula having a forward tip is coupled to the forward end of the barrel and is in fluid communication with the reservoir. The syringe assembly also includes a plunger having a first end with a stopper positioned in the reservoir and a second end having a thumb pad for receiving medicament delivery pressure for causing the plunger to move within the reservoir to cause the medicament to be expelled from the reservoir.

The medical device also includes an outer shield having a forward end and a rear end movable on the barrel from a first position, in which the forward tip of the needle cannula extends past the forward end of the outer shield, and a second position in which the forward tip of the needle cannula is between the forward and rear ends of the outer shield. An urging member is connected to the outer shield. The plunger interacts with the outer shield to charge the urging member with an urgency when the needle cannula is inserted into the patient and the plunger is moved into the barrel to expel medicament from the reservoir into the patient. The front of the outer shield is movable by the urgency of the charged urging member toward the second position upon removal of the needle cannula from the patient.

The outer shield has a first wall at its forward end, and a rear wall at its rear end. The needle cannula extends through a hole defined through the front wall when the outer shield is in the first position. The rear wall is arranged between the rear end of the barrel and the thumb pad. The outer shield further includes first and second legs, each being connected between the front and rear walls of said outer shield.

The rear end of the outer shield is movable by the plunger while the front end is held in a fixed position against a patient's skin during medicament delivery. This movement of the rear end relative to the front end charges the urging member with an urgency, i.e. a force accumulates in the urging member. In response to the relative movement between the front end and the second end, the first and second legs flex radially outward. To facilitate the bulging movement, each of the first and second legs includes at least one living hinge between the ends thereof. The at least one living hinge allows the first and second legs to flex radially outward when the rear end of the outer shield is moved toward the front end. The urging member comprises an elastic band arranged around the outer shield which expands when the first and second legs flex outward. The medical device may further include a cylindrical sheath having a front end and a rear end and arranged radially inside the first and second legs. The front end of the sheath is connected proximate the front end of the outer shield such that the inner sheath surrounds the needle cannula in the second position. The elastic band is arranged axially between the front and rear ends of the inner sheath.

As an alternative, the urging member may be a spring acting between the front and rear ends of the outer shield.

The medical device may include a flap arranged proximate the front wall of the outer shield to cover the hole at the front wall of the outer shield when the outer shield is in the second position.

The outer shield may comprise a latching mechanism for preventing the rear end of the outer shield from moving rearward relative to the barrel after the plunger is depressed to a fully inserted position in which the stopper is proximate the front end of the barrel.

In a further alternative embodiment, each of the first and second legs includes a forward portion and a rearward portion. The rearward portion moves relative to the forward portion when the rear end of the outer shield is moved relative to the front end. In this embodiment, the urging member comprises a spring acting between the front and rear portions of each of said first and second legs.

The front wall may be held askew in the second position such that the needle cannula is out of alignment with the hole through said front wall.

The barrel of the syringe may be made of glass or plastic.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings, wherein like reference characters denote similar elements throughout the several views:

FIG. 1 is a perspective view of a medical device according to an embodiment of the present invention;

FIG. 2 is a longitudinal sectional view of the medical device of FIG. 1;

FIG. 3 is a enlarged sectional view of the front end of the medical device of FIG. 1;

FIG. 4 is a longitudinal sectional view of the medical device of FIG. 1 after the plunger is moved to a fully inserted position;

FIG. 5 is an enlarged side view of the front end of the medical device in the position shown in FIG. 4;

FIG. 6 is a partial sectional side view of medical device of FIG. 1 after use thereof;

FIG. 7 is a longitudinal sectional view of a medical device according to a further embodiment of the present invention;

FIG. 8 is a longitudinal sectional view of a medical device according to yet another embodiment of the present invention; and

FIG. 9 is an enlarged sectional view of the medical device of FIG. 8 after use thereof.

### DETAILED DESCRIPTION OF THE PRESENTLY PREFERRED EMBODIMENTS

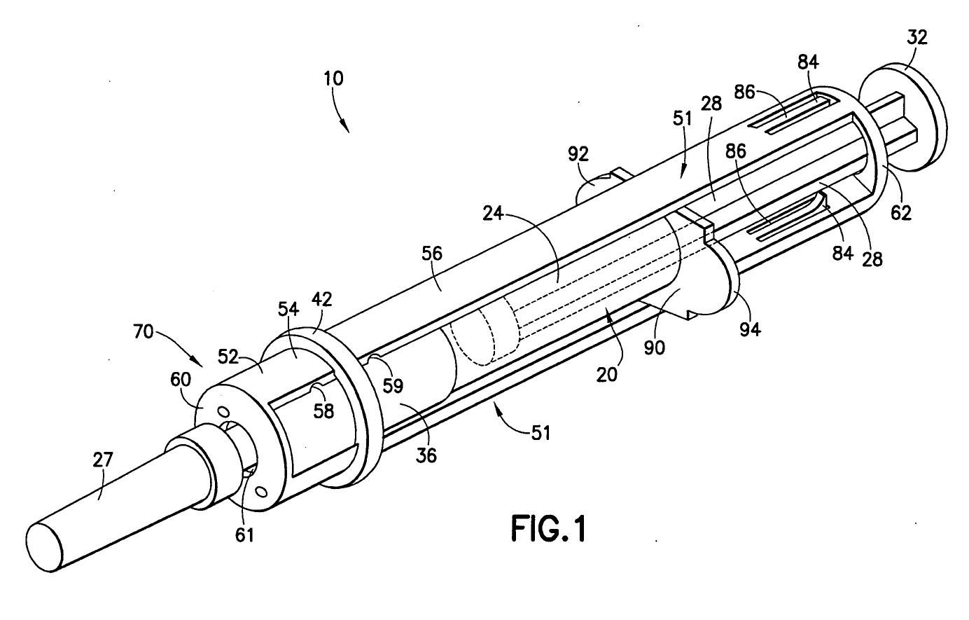

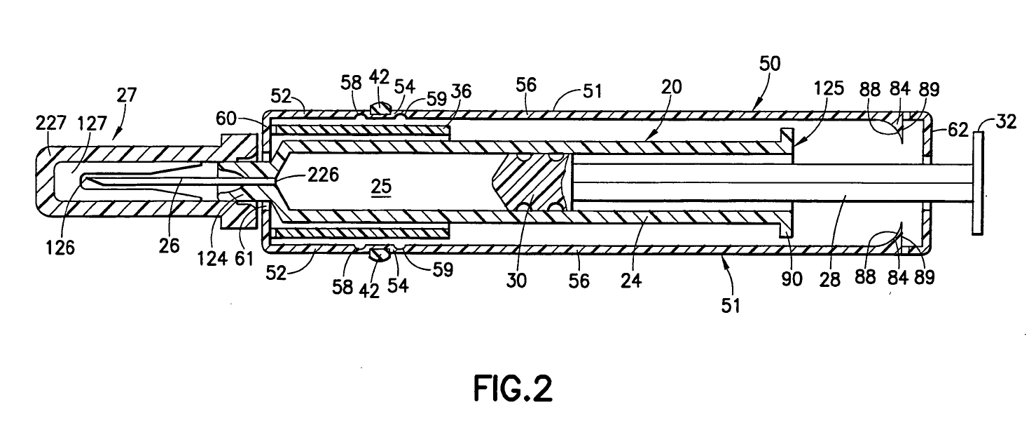

FIGS. 1 and 2 show a medical device 10 for delivery of a medicament into a patient constructed in accordance with an embodiment of the present invention. As used herein, the term “medicament” is intended to refer to any drug substance, vaccine, or other liquid substance that is injected into a patient. The medical device 10 includes a syringe assembly 20 which can be prefilled with the medicament to be delivered, and a safety shield assembly 70 which partially surrounds the syringe 20.

The syringe assembly 20 includes a cylindrical barrel 24 having a front end 124, and a rear end 125, with a flange 90 formed at the rear end. The barrel 24 defines a reservoir 25 within which the medicament may be held prior to use of the medical device 10. The syringe assembly 20 also includes a needle cannula 26 having a forward tip 126 and a rearward end 226 in fluid communication with the reservoir 25. The needle cannula 26 may be permanently connected to a front end of the barrel 24 using an adhesive, glue, interference fit or other known or hereafter developed material or technique, or it may be detachable from the barrel 24 such as, for example, using a luer-type connection. The barrel 24 is preferably made of a molded plastic. Alternatively, the barrel 24 may be formed from glass. A plunger rod 28 has a first end inserted in the barrel 24 with a stopper or piston 30 arranged on the first end that is movable with the plunger rod 28 within the barrel 24. A second end of the plunger rod 28 includes a thumb pad 32 used for receiving pressure from the user's thumb for moving the piston 30 into and within the barrel 24. Finger rests 92, 94 (see FIG. 1) are arranged on a flange 90 at the rear end 125 of the barrel 24 to provide ergonomic grips for holding the medical device 10 during insertion of the needle cannula 26 and during the application of medicament delivery force to the thumb pad 32. Although the finger rests 92, 94 are depicted as part of the flange 90, other designs may be used such as, for example, individual radial projections. As further shown in FIGS. 1 and 2, a removable needle shield 27 is disposed over the needle cannula 26 on the front end of the barrel 24 to protect the needle from damage during handling of the syringe assembly, and to protect users from being stuck by the needle prior to its intended use. The needle shield 27 preferably includes a pliable part 127 and a rigid part 227.

The safety shield assembly 70 includes an outer shield 50 and an inner sheath 36. The outer shield 50 includes a front wall 60 having a hole 61 through which the needle cannula 26 extends, and a rear wall 62 connected to the front wall 60 by two legs 51. When the outer shield 50 is in the position depicted in FIG. 2, the rear wall is positioned between flange 90 and thumb pad 32. Although two legs 51 are shown, any number of legs may be used, provided that access to the finger rests 92, 94 is not obstructed. Each of the legs 51 includes three sections: a forward section 52, a central section 54, and a rear section 56. An annular urging member 42 surrounds the outer shield in the area of the central section 54. A living hinge 58 is disposed between the forward section 52 and the central section 54 and another living hinge 59 is disposed between the central section 54 and the rear section 56.

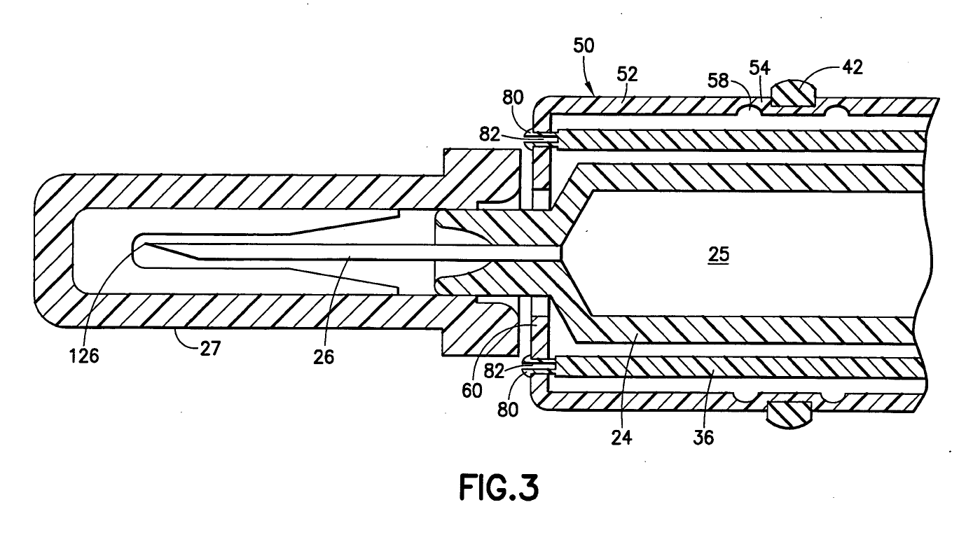

The inner sheath 36 is a cylindrical-shaped element connected to the outer shield 50 at the forward wall 60 which annularly surrounds the barrel. The connection may be made using adhesive or glue or it may comprise a press-fit connection as shown in FIG. 3. For the press-fit connection, latching projections 80 are inserted into holes 82 formed in the forward wall 60 of the outer shield 50.

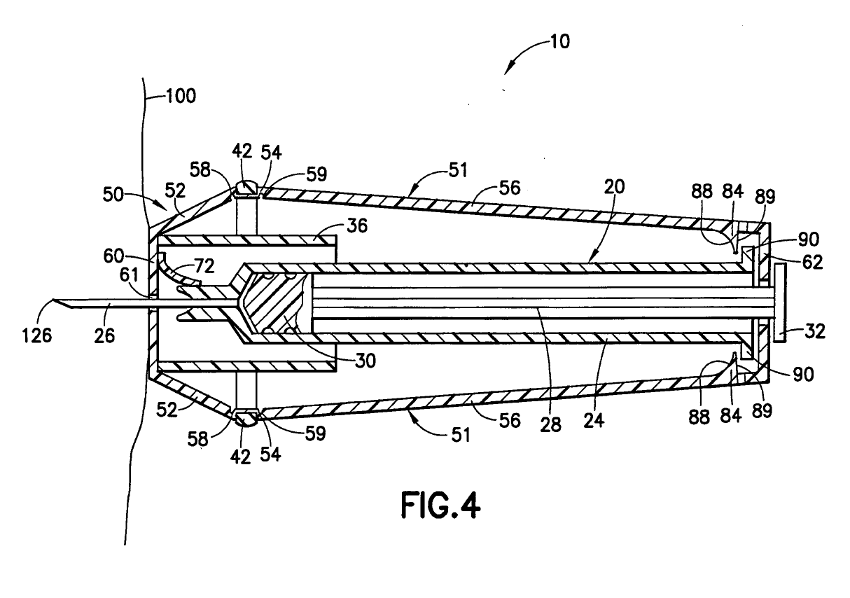

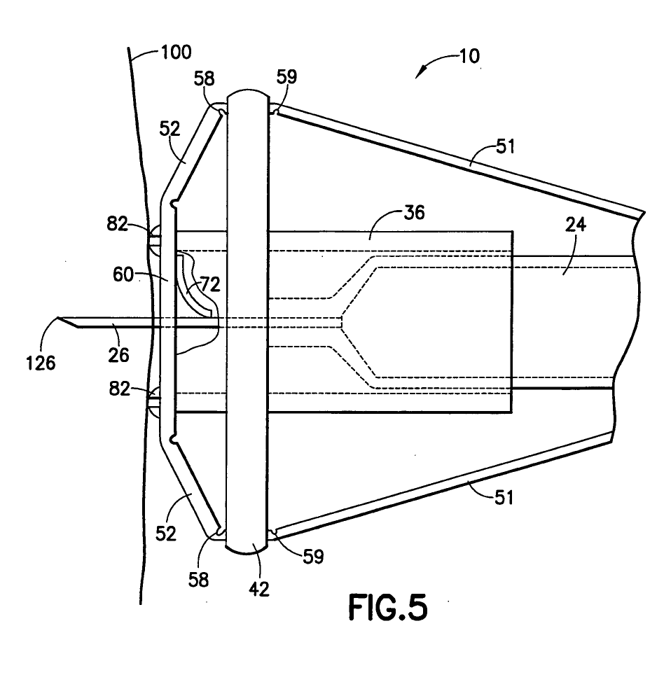

FIGS. 4 and 5 show the medical device 10 after the needle tip 126 is inserted into a patient's skin 100 and after the plunger 28 is moved to a fully inserted position in which the piston 30 is proximate the front end of the barrel 24. In this position, the forward wall 60 of the outer shield 50 abuts the skin 100 of the patient. As the plunger 28 is pushed further into the barrel 24, the rear wall 62 is pushed toward the patient's skin 100 due to contact with the thumb pad 32 while the front wall 60 is prevented from moving forward as a result of being obstructed from the patient's skin. This obstruction causes the legs 51 to flex radially outward at the living hinges 58, 59. To achieve this result, the outer shield 50 is preferably made of a substantially rigid plastic such that the legs 51 bend only at the living hinges 58, 59 along the length of the legs 51. When the legs 51 flex as shown in FIGS. 4 and 5, the urging member 42 is stretched. The urging member 42 is preferably configured as a band of elastic material such that when the legs 51 flex, the urging member is stretched, i.e., it accumulates a force, which urges the legs radially inward. To prevent the legs 51 from flexing inward, the living hinges 58, 59 are designed to facilitate only the radially outward flexure of the legs 51. Alternatively or in addition, the inner sheath 36 may be sufficiently strong to prevent inward flexure of the legs.

As shown in FIGS. 1 and 4, the outer shield 50 may include latching projections 84 arranged on flexible arms 86 proximate the rear end of the barrel 24. The latching projections 84 include an angled or curved forward facing edge 88 and a straight rear facing edge 89. The curved forward facing edge 88 facilitates movement of the latching projections in a forward motion over the flange 90 toward the needle cannula 26, which occurs during movement of the thumb pad 32, and contact with rear wall 62, during medicament delivery. The rear facing edge 89 prevents relative rearward motion of the outer shield 50 with respect to the barrel due to abutment of the rear facing edge 89 and the flange 90. This feature is described more fully below. As an alternative to arranging the projections 84 on the flexible arms 86, the projections may be arranged on, or connected to, the rear wall 62.

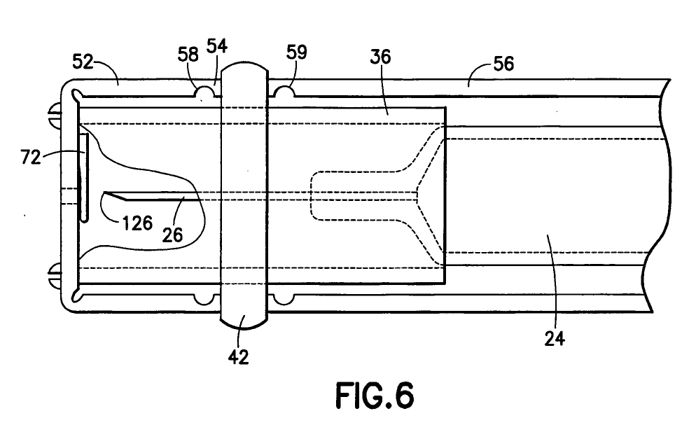

When the full dose of medicament is delivered, the needle cannula is removed from the patient. As the needle cannula 26 is removed, the patient's skin no longer blocks the movement of the front wall 60. The urgency force stored in the urging member 42 forces the legs radially inward until the inner sides of the legs 51 abut the outer surface of the inner sheath 36 as shown in FIG. 6. In this position, and due to the engagement of the rear facing edge 89 and the flange 90, the front wall 60 of the outer shield 50 extends past the forward tip 126 of the needle cannula 26 and the inner sheath 36 circumferentially surrounds the needle cannula 26, thereby protecting against inadvertent contact of the used needle cannula 26 with a user's finger after use. To prevent the needle cannula 26 from re-emerging from the hole 61 in the front wall 60, a flap 72 may be arranged within the inner sheath 36 which covers the hole 61 after the forward tip 126 of the needle cannula enters the inner sheath (see FIGS. 4-6).

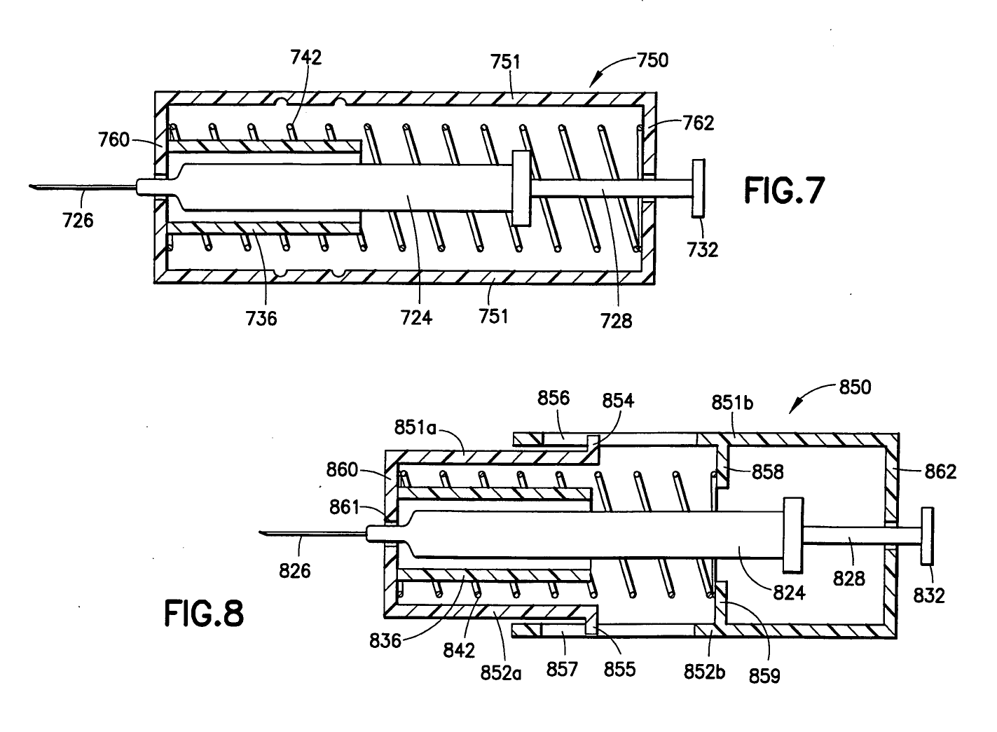

FIG. 7 shows an alternate embodiment in which an urging member 742 is a coil spring arranged between the forward wall 760 and the rear wall 762 of an outer shield 750 which includes legs 751 similar to the legs 51 of the embodiment of FIGS. 1-6. In this embodiment, as the plunger 728 moves into the barrel 724, the coil springs are charged with an urgency that acts on the front wall 760 when the needle cannula is withdrawn from a patient to push the front wall 760 and inner sheath 736 connected thereto over the needle cannula 726.

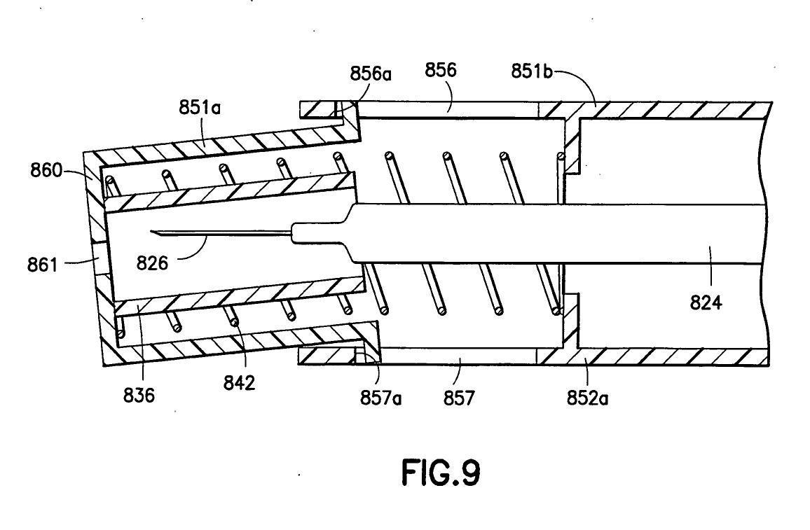

FIG. 8 shows yet another embodiment in which an outer shield 850 includes a front wall 860 connected to front legs 851a, 852a and a rear wall 862 connected to rear legs 851b, 852b. Front legs 851a, 852a include pins 854, 855, respectively, which engage slots 856, 857 in rear legs 851b, 852b. Urging members 842 are respectively arranged between the front wall 860 and projections 858, 859 on the rear legs 851b, 852b. Alternatively, the urging members 842 may be made longer so that they extend between the front wall 860 and the rear wall 862, thereby obviating the need for the projection 858, 859. When the needle cannula 826 is inserted in a patient and the plunger 828 is pushed into the barrel 824, the rear wall 862 and rear legs 851b, 852b move forward relative to the front wall 860 and the front legs 851a, 851b. This relative movement compresses the urging members 842, thereby developing an urgency which pushes the front wall 860 and inner sheath 836 over the needle cannula 826 when the needle cannula 826 is withdrawn from the patient. To prevent the needle from reentering the hole 861 in the front wall 860, the front end 857a of slot 857 is offset relative to the front end 856a of slot 856 such that the front wall 860 and front legs 851a, 852a are held askew relative to the longitudinal axis of the barrel 824 when the front wall 860 is moved over the needle cannula 826 as shown in FIG. 9.

A description of an exemplary usage of the medical device 10 of the present invention will now be provided. It should be understood by a person of ordinary skill in the art that the following description is provided as an illustrative and non-limiting example. The health care professional receives the inventive medical device 10 prefilled with a desired single dosage of a medicament. Immediately prior to use, the needle shield 27 is removed and the needle cannula 26 and forward tip 126 are exposed. The health care professional pierces the patient's skin with the forward tip 126 of the needle cannula 26 and depresses the thumb pad 32 to cause the plunger rod 28 and piston 30 to move within the reservoir 25. As the plunger rod 28 and piston are caused to move into the reservoir 25, the medicament is caused to be expelled from the reservoir, through the needle cannula 26, and into the patient.

During movement of the plunger rod 28 to deliver the medicament, the thumb pad interacts with the rear wall 62 of the outer shield 50 and the rear wall moves with the thumb pad 32 so that the latching projections 84 are positioned on the forward side of the flange 90, as shown in FIG. 4, while the front wall 60 of the outer shield 50 is held against the patient's skin 100. Legs 51 connecting the front wall 60 and rear wall 62 flex outward stretching an elastic band 42 which surrounds the outer shield 50. When the medicament is completely expelled from the reservoir (i.e., the dose has been completely administered), the needle cannula 26 is removed from the patient. Due to the engagement of the straight rear facing edge 89 with the flange 90, the front wall is urged in a forward direction toward and over the forward tip 126 of the needle cannula 26 by the urgency of the elastic band 42 to a second position of the outer shield. When in the second position, the forward tip 126 of the needle cannula 24 is located a sufficient distance between the front wall 60 and rear wall 62 of the outer shield 50, thus preventing undesired and inadvertent contact of a user's finger with the contaminated forward tip 126. The inner sheath 36 circumferentially surround the needle cannula 26 in the second position. The used medical device 10 may then be disposed of in a suitable sharps disposal container.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements which perform substantially the same function in substantially the same way to achieve the same results are within the scope of the invention. Moreover, it should be recognized that structures and/or elements shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A medical device for delivering a medicament to a patient, comprising:
a syringe assembly comprising:
a barrel having a forward end and a rear end and defining a reservoir within which the medicament may be contained;
a needle cannula having a forward tip and being coupled to said forward end of said barrel and in fluid communication with said reservoir; and
a plunger having a first end with a stopper positioned in said reservoir and a second end having a thumb pad for receiving medicament delivery pressure for causing said plunger to move within said reservoir to cause the medicament to be expelled from said reservoir;
an outer shield having a forward end and a rear end movable on said barrel from a first position, in which said forward tip of said needle cannula extends past said forward end of said outer shield, and a second position in which said forward tip of said needle cannula is between said forward end and said rear end of said outer shield; and
an urging member connected to said outer shield, wherein said plunger interacts with said outer shield to charge said urging member with an urgency when said needle cannula is inserted into the patient and said plunger is moved into said barrel to expel medicament from said reservoir into the patient, said front end of said outer shield being movable by the urgency of said urging member toward said second position upon removal of said needle cannula from the patient.

2. The medical device of claim 1, wherein said outer shield comprises a first wall at said forward end of said outer shield and a rear wall at said rear end of said outer shield, said needle cannula extending through a hole defined through said front wall in said first position, said rear wall being arranged between said rear end of said barrel and said thumb pad.

3. The medical device of claim 2, wherein said outer shield comprises first and second legs, each of said first and second legs being connected between said front wall and said rear wall.

4. The medical device of claim 3, further comprising a sheath connected to said outer shield proximate said front end of said outer shield for surrounding said needle cannula when said outer shield is in said second position.

5. The medical device of claim 3, wherein said rear end of said outer shield is movable by said plunger while said front end is held in a fixed position against the patient's skin during medicament delivery to charge said urging member with the urgency.

6. The medical device of claim 5, wherein said first and second legs flex radially outward when said rear end of said outer shield is moved relative to said front end.

7. The medical device of claim 6, wherein each of said first and second legs includes at least one living hinge between the ends thereof, the at least one living hinge allowing said first and second legs to bend about said living hinges and flex radially outward when said rear end of said outer shield is moved relative to said front end.

8. The medical device of claim 6, wherein said urging member comprises an elastic band arranged around said outer shield which stretches when said first and second legs flex outward to charge said urging member with said urgency.

9. The medical device of claim 8, further comprising a cylindrical sheath having a front end and a rear end and arranged radially inside said first and second legs, said front end of said sheath being connected proximate said front end of said outer shield such that said inner sheath surrounds said needle cannula in said second position, said elastic band being arranged axially between said front and rear ends of said inner sheath.

10. The medical device of claim 6, wherein said urging member is a spring acting between said front and rear ends of said outer shield.

11. The medical device of claim 2, further comprising a flap arranged proximate said front wall of said outer shield, said flap being resiliently loaded to move from an uncovered position in which said hole is uncovered when said outer shield is in said first position to a covered position for covering said hole in said front wall when said outer shield is in said second position.

12. The medical device of claim 1, wherein said rear end of said outer shield is movable by said plunger while said front end of said outer shield is held in a fixed position against a patient's skin during medicament delivery to charge said urging member with the urgency.

13. The medical device of claim 12, wherein said outer shield comprises a latching mechanism for preventing said rear end of said outer shield from moving rearward relative to said barrel after said plunger is depressed to a fully inserted position in which said stopper is proximate said front end of said barrel.

14. The medical device of claim 5, wherein each of said first and second legs comprises a forward portion and a rearward portion, said rearward portion movable relative to said forward portion when said rear end of said outer shield is moved relative to said front end.

15. The medical device of claim 14, wherein said urging member comprises a spring acting between said front and rear portions of each of said first and second legs.

16. The medical device of claim 15, wherein said front wall and front portions of said first and second legs are held askew in said second position such that said needle cannula is out of alignment with said hole through said front wall.

17. The medical device of claim 2, wherein said front end of said outer shield is held askew in said second position such that said needle cannula is out of alignment with said hole in said front end.

18. The medical device of claim 1, wherein said barrel comprises glass.

19. The medical device of claim 1, wherein said barrel comprises plastic.

20. A combination comprising a medical syringe with a shielding system, said medical syringe comprising a barrel having a forward end and a rear end and defining a reservoir within which the medicament may be contained, a needle cannula having a forward tip and being coupled to said forward end of said barrel and in fluid communication with said reservoir, and a plunger having a first end with a stopper positioned in said reservoir and a second end having a thumb pad for receiving medicament delivery pressure for causing said plunger to move within said reservoir to cause the medicament to be expelled from said reservoir,
said shielding system comprising an outer shield having a forward end and a rear end movable on said barrel from a first position, in which said forward tip of said needle cannula extends past said forward end of said outer shield, and a second position in which said forward tip of said needle cannula is between said forward end and said rear end of said outer shield, and
an urging member connected to said outer shield, wherein said plunger interacts with said outer shield to charge said urging member with an urgency when said needle cannula is inserted into the patient and said plunger is moved into said barrel to expel medicament from said reservoir into the patient, said front end of said outer shield being movable by the urgency of said urging member toward said second position upon removal of said needle cannula from the patient.

21. The combination of claim 20, wherein said outer shield comprises a first wall at said forward end of said outer shield and a rear wall at said rear end of said outer shield, said needle cannula extending through a hole defined through said front wall in said first position, said rear wall being arranged between said rear end of said barrel and said thumb pad.

22. The medical device of claim 21, wherein said outer shield comprises first and second legs, each of said first and second legs being connected between said front wall and said rear wall.

23. The medical device of claim 22, further comprising a sheath connected to said outer shield proximate said front end of said outer shield for circumferentially surrounding said needle cannula when said outer shield is in said second position.

24. The medical device of claim 22, wherein said rear end of said outer shield is movable by said plunger while said front end is held in a fixed position against the patient's skin during medicament delivery to charge said urging member with the urgency.

25. The medical device of claim 24, wherein said first and second legs flex radially outward when said rear end of said outer shield is moved relative to said front end.

26. The medical device of claim 25, wherein each of said first and second legs includes at least one living hinge between the ends thereof, the at least one living hinge allowing said first and second legs to bend about said living hinges and bulge radially outward when said rear end of said outer shield is moved relative to said front end.

27. The medical device of claim 25, wherein said urging member comprises an elastic band arranged around said outer shield which stretches when said first and second legs bulge outward to charge said urging member with the urgency.

28. The medical device of claim 27, further comprising a cylindrical sheath having a front end and a rear end and arranged radially inside said first and second legs, said front end of said sheath being connected proximate said front end of said outer shield such that said inner sheath surrounds said needle cannula in said second position, said elastic band being arranged axially between said front and rear ends of said inner sheath.

29. The medical device of claim 25, wherein said urging member is a spring acting between said front and rear ends of said outer shield.

30. The medical device of claim 21, further comprising a flap arranged proximate said front wall of said outer shield, said flap being resiliently loaded to move from an uncovered position in which said hole is uncovered when said outer shield is in said first position to a covered position for covering said hole in said front wall when said outer shield is in said second position.

31. The medical device of claim 20, wherein said rear end of said outer shield is movable by said plunger while said front end of said outer shield is held in a fixed position against a patient's skin during medicament delivery to charge said urging member with the urgency.

32. The medical device of claim 31, wherein said outer shield comprises a latching mechanism for preventing said rear end of said outer shield from moving rearward relative to said barrel after said plunger is depressed to a fully inserted position in which said stopper is proximate said front end of said barrel.

33. The medical device of claim 24, wherein each of said first and second legs comprises a forward portion and a rearward portion, said rearward portion movable relative to said forward portion when said rear end of said outer shield is moved relative to said front end.

34. The medical device of claim 33, wherein said urging member comprises a spring acting between said front and rear portions of each of said first and second legs.

35. The medical device of claim 34, wherein said front wall and front portions of said first and second legs are held askew in said second position such that said needle cannula is out of alignment with said hole through said front wall.

36. A medical device for delivering a medicament to a patient, comprising:
a syringe assembly comprising:
a barrel having a forward end and a rear end and defining a reservoir within which the medicament may be contained;
a needle cannula having a forward tip and being coupled to said forward end of said barrel and in fluid communication with said reservoir; and
a plunger having a first end with a stopper positioned in said reservoir and a second end having a thumb pad for receiving medicament delivery pressure for causing said plunger to move within said reservoir to cause the medicament to be expelled from said reservoir;
an outer shield having a forward end and a rear end movable on said barrel from a first position, in which said forward tip of said needle cannula extends past said forward end of said outer shield, and a second position in which said forward tip of said needle cannula is between said forward and rear ends of said outer shield;
an urging member connected to said outer shield; and
means for charging said urging member with an urgency when said needle cannula is inserted into the patient and said plunger is moved into said barrel to expel medicament from said reservoir into said patient, said front of said outer shield being movable by the urgency of said urging member toward said second position upon removal of said needle cannula from the patient.
